(19) United States
(12) Patent Application Publication
(10) Pub. No.: US 2009/0157094 A1
(43) Pub. Date: Jun. 18, 2009

(54) DEVICE FOR SUPERFICIAL ABRASIVE TREATMENT OF THE SKIN

(75) Inventors: Yehoshua Yeshurun, Haifa (IL); Yotam Levin, Nes Ziona (IL); Meir Hefetz, Mizpeh Harashim (IL); Yoel Sefi, Merom Hagalil (IL); Gilad Lavi, Rishon Lezion (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: NanoPass Technologies Ltd., Nes Ziona (IL)

(21) Appl. No.: 12/159,210

(22) PCT Filed: Jan. 14, 2007
(86) PCT No.: PCT/IL07/00053

§ 371 Date: Oct. 5, 2008

Related U.S. Application Data
(60) Provisional application No. 60/758,262, filed on Jan. 12, 2006.

(51) Int. Cl. A61B 19/00; A61B 17/20

(52) U.S. Cl. 606/131; 604/46

(57) ABSTRACT

A device (100, 200) for superficial abrasive treatment of the skin includes a skin interface element (102, 202) with a number of protrusions (106, 206) projecting to a height above a substrate (104, 204) of no greater than 200 microns. A vibration generating mechanism (108, 208) is mechanically linked to the skin interface element (102, 202) so as to generate vibratory motion of the skin interface element (102, 202).

### FIELD AND BACKGROUND OF THE

The present invention relates to a cosmetic or medical tool which includes micro protrusion elements combined with a mechanism for generating vibrations. The tool may be used to implement a method for treating human skin to enhance the penetration of medical or cosmetic compounds, or to provide other benefit such as skin rejuvenation, smoothening, resurfacing, peeling, improved healing or regeneration or the like.

There are a wide range of beneficial cosmetic or medical effects which are associated with superficial abrasive treatment of the skin, sometimes referred to as dermabrasion or micro-dermabrasion. In many applications, the abrasive treatment may be performed by the user or other medically untrained personnel using simple “rough” devices such as sandpaper which are moved manually across the skin. Other devices have proposed the use of orderly arrangements of projections from a surface to provide the abrasive features. Examples of such devices are disclosed in US Patent Application Publication Nos. 2004/0064087 and 2004/0097967. In each case, motion of the projections across the skin is induced by manual displacement of the device, and as such is highly user-dependent, tending to produce irregular, uncontrolled and variable results. Furthermore, these approaches are not well suited to treatment of a localized region of skin without affecting also the peripheral (surrounding) region.

Other devices proposed for similar purposes include various devices with rotating abrasive disks and the like. Rotating disks tend to produce very non-uniform results, with the outer periphery being exposed to much more motion of the abrasive disk than the inner part (i.e., closer to the center).

Some microdermabrasion devices utilize particles that bombard the skin in order to achieve exfoliating results. These again are variable in results and difficult to control. They are also prone to other potential damages, such as the undesired inhalation of the particles during treatment. These devices are typically expensive, require power sources, must be operated by trained technicians and are therefore generally unsuitable for home use.

There is therefore a need for a device for superficial abrasive treatment of the skin which would provide reliable and more uniform results when operated by a user without special training without relying upon skilled manual motion of the user to achieve abrasion. It would also be advantageous to provide an easy-to-use inexpensive device for superficial abrasive treatment of the skin which is suited for treatment of a localized region without affecting the surrounding skin, that requires little training, does not require an external power source, and is therefore highly suited for home or consumer use.

### SUMMARY OF THE INVENTION

The present invention relates to a cosmetic or medical tool which includes micro protrusion elements combined with a mechanism for generating vibrations. The tool may be used to implement a method for treating human skin to enhance the penetration of medical or cosmetic compounds, or to provide other benefit such as skin rejuvenation, smoothening, resurfacing, peeling, improved healing or regeneration or the like.

According to the teachings of the present invention there is provided, a device for superficial abrasive treatment of the skin comprising: (a) a skin interface element including a substrate provided with a plurality of protrusions, the protrusions projecting to a height above the substrate of no greater than 200 microns; and (b) a vibration generating mechanism mechanically linked to the skin interface element so as to generate vibratory motion of the skin interface element.

According to a further feature of the present invention, the protrusions have a height above the substrate of between about 20 microns and about 100 microns.

According to a further feature of the present invention, the protrusions are arranged in a two-dimensional array.

According to a further feature of the present invention, the protrusions have a shape selected from the group comprising: pyramids, cones and rods.

According to a further feature of the present invention, the protrusions are integrally formed with the substrate.

According to a further feature of the present invention, the protrusions and the substrate are formed from a single crystal of material.

According to a further feature of the present invention, the protrusions and the substrate are formed from a unitary block of material processed primarily by wet etching techniques.

According to a further feature of the present invention, the protrusions are formed from a material selected from the group consisting of: silicon, a polymer, a metal, a metal alloy, and a ceramic material.

According to a further feature of the present invention, the vibration generating mechanism includes a motor configured for rotating an eccentric weight about an axis.

According to a further feature of the present invention, the vibration generating mechanism is configured to generate vibratory motion corresponding to an orbital motion in a plane of the substrate.

According to a further feature of the present invention, the vibration generating mechanism is configured to generate vibratory motion having a non-zero component perpendicular to a plane of the substrate.

According to a further feature of the present invention, the vibration generating mechanism is configured to generate vibratory motion having a frequency in the range between 50 Hz and 200 Hz, and most preferably in the range of 140 Hz±25 Hz.

According to a further feature of the present invention, there is also provided a pressure-limiting switch arrangement, associated with the skin interface element and responsive to contact pressure of the skin interface element above a given limit to interrupt operation of the vibration generating mechanism.

According to a further feature of the present invention, there is also provided a housing, wherein the skin interface element is resiliently mounted relative to the housing, and wherein the vibration generating mechanism is mechanically linked to the skin interface element so as to generate vibratory motion of the skin interface element relative to the housing.

There is also provided according to the teachings of the present invention a method for superficial abrasive treatment of the skin comprising the steps of: (a) bringing into contact with the skin a skin interface element including a substrate provided with a plurality of protrusions, the protrusions projecting to a height above the substrate of no greater than 200 microns; and (b) employing a vibration generating mechanism mechanically linked to the skin interface element to generate vibratory motion of the skin interface element so that the protrusions perform superficial abrasive treatment of the skin.

### BRIEF DESCRIPTION OF THE DRAWINGS

The invention is herein described, by way of example only, with reference to the accompanying drawings, wherein:

FIG. 1 is a schematic representation of a device for superficial abrasive treatment of the skin, constructed and operative according to the teachings of the present invention;

FIG. 2 is a schematic representation of a variant of the device of FIG. 1 illustrating an additional pressure-limiting switch arrangement;

FIG. 3 is an isometric view of a proposed implementation of the device of FIG. 1;

FIG. 4 is a partially cut-away isometric view of the implementation of FIG. 3;

FIG. 5 is a side cross-section view of the implementation of FIG. 3;

FIG. 6 is an enlarged view of the region of FIG. 5 showing a skin contact interface;

FIG. 7 is an isometric view of the implementation of FIG. 3 during replacement of a disposable portion of the device;

FIG. 8 is an isometric view of a first implementation of a skin interface element from the implementation of FIG. 3;

FIG. 8A is an enlarged view of a single micro-protrusion from the skin interface element of FIG. 8;

FIG. 9 is an isometric view of a second implementation of a skin interface element from the implementation of FIG. 3;

FIG. 9A is an enlarged view of a single micro-protrusion from the skin interface element of FIG. 9;

FIG. 10 is an isometric view of an alternative implementation of the device of FIG. 1;

FIG. 11 is an enlarged view of a part of FIG. 10 showing a skin interface element;

FIG. 12 is an enlarged view of a small region of FIG. 11 showing the structure of the protrusions; and

FIG. 13 is a partially cut-away isometric view of a part of the device of FIG. 10.

### DESCRIPTION OF THE PREFERRED EMBODIMENTS

The present invention is a device for superficial abrasive treatment of the skin.

The principles and operation of devices according to the present invention, and the corresponding method, may be better understood with reference to the drawings and the accompanying description.

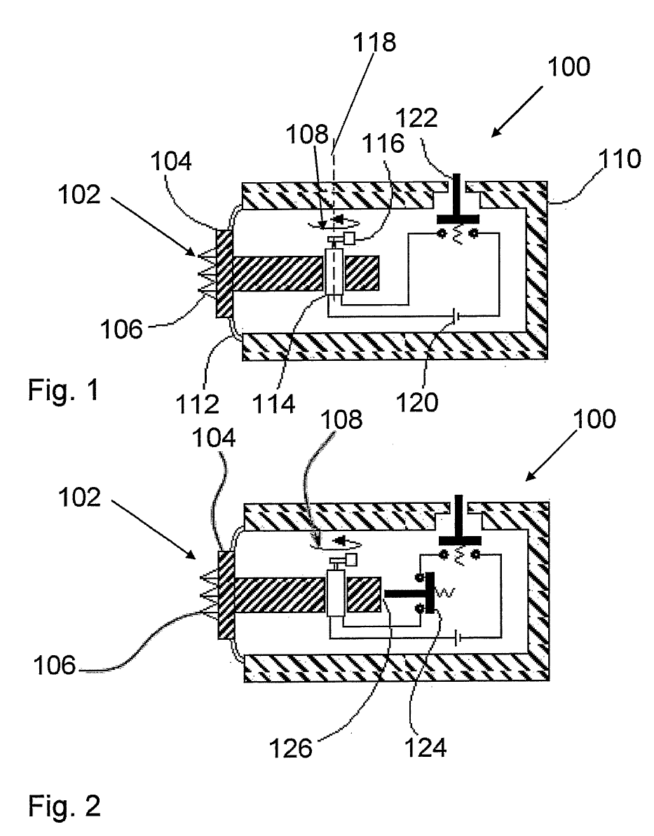

Referring now to the drawings, FIG. 1 illustrates schematically a device, generally designated 100, constructed and operative according to the teachings of the present invention, for superficial abrasive treatment of the skin. Generally speaking, device 100 includes a skin interface element 102 having a substrate 104 from which project a plurality of protrusions 106. Protrusions 106 project to a height above the substrate of no greater than 200 microns. A vibration generating mechanism 108 is mechanically linked to skin interface element 102 so as to generate vibratory motion of skin interface element 102.

The combination of the vibratory motion together with the protrusions 106 achieves superficial abrasion of the outer surface of the skin, typically only at the level of the stratum corneum (SC) or, in relevant specific cases, the epidermis, and is thus effective for a wide range of cosmetic and medical applications as are known for micro-dermabrasion or dermabrasion. At the same time, the use of vibration generating mechanism 108 ensures that operation of the device is not critically dependent upon the manner in which the device is moved across the skin surface, thereby facilitating more uniform and reliable results than conventional manually-reciprocated devices. These and other advantages of the present invention will become clearer from the following description.

Before addressing the features of the present invention in more detail, it will be helpful to define certain terminology as used herein in the description and claims. Firstly, reference is made to “vibration” and “vibratory motion”. These terms are used herein in the description and claims to refer to any repetitive oscillatory motion about a mean position in one or more dimension. These vibrations may be linear (i.e., one dimensional) or orbital (i.e., circular or elliptical), or may have a more complex form such as results from, for example, differing vibration frequencies in two perpendicular directions. The vibratory motion is preferably translational rather than rotating. In other words, the motion of all parts of skin interface element 102 is preferably roughly the same so that the entire element vibrates to-and-fro, or orbits, without rotation of skin interface element 102.

Typically, the vibrations are actually oscillating forces applied to the skin interface element 102 and the amplitude of the vibrations varies, depending upon the damping effect of engagement with the skin.

The term “superficial” is used herein in the description and claims to refer to abrasion of the skin which does not extend to a depth of more than 200 microns. For cosmetic applications, the superficial abrasion is preferably kept to a depth less than 100 microns, thereby avoiding fully breaching the upper barrier layers of the skin (SC and upper epidermal layers), so as to minimize pain, damage to the viable dermis, and other adverse effects. For this purpose, penetration into the layers of the skin is preferably limited to less than 100 microns, and most preferably less than about 70 microns. The actual height of the protrusions 106 above the surface of substrate 104 may be somewhat larger than the desired penetration depth, since the entire height does not typically penetrate. Preferred heights for protrusions 106 are thus typically in the range of about 20 microns to about 100 microns, and most preferably 60 microns±20 microns. For medical applications, on the other hand, for example where permeability of the skin is to be enhanced for the transfer of substances through the skin, penetration depths in excess of 100 microns are typically indicated. In this case, protrusions of height in the range of 100 microns up to 200 microns are typically used, although taller protrusions up to about 500 microns could also be useful in certain applications.

The term “protrusions” is used to refer to any repetitive structure of projecting features which project from the surface of substrate 104. The protrusions may be any shape, pointed or blunt-ended, rounded in cross-section or with lateral cutting edges, hollow or solid. Non limiting examples of particularly preferred forms of protrusion include: symmetrical or asymmetric pyramids of polygonal base, pointed or truncated cones, and cylindrical or polygonal rods.

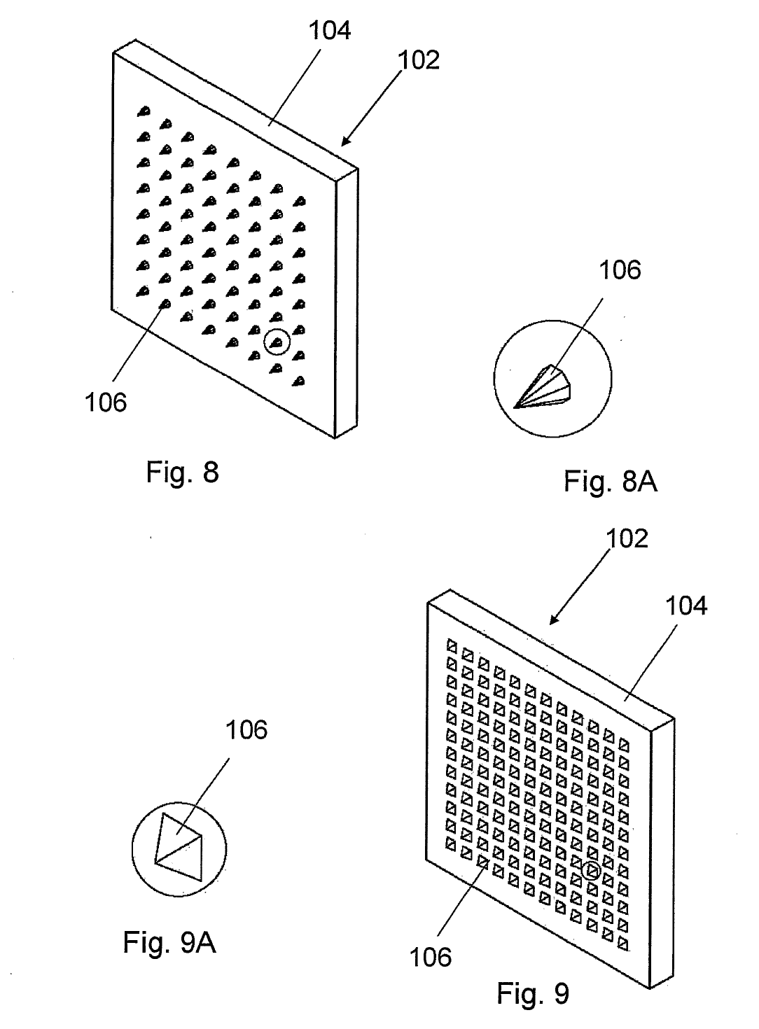

Turning now to the features of the present invention in more detail, protrusions 106 are preferably arranged in a two-dimensional array, and typically in a rectangular array. Two non-limiting examples are illustrated in FIGS. 8 and 9. Most preferably, dimensions of the two-dimensional array are at least 8×8, and more preferably at least 10×10, corresponding to a total of at least 100 protrusions. Typically, several hundred protrusions are provided on an area of less than one square centimeter. In the examples illustrated here, FIG. 8 (enlarged in FIG. 8A) shows octagonal pyramid protrusions, while FIG. 9 (enlarged in FIG. 9A) shows square pyramidal protrusions. In either case, the protrusions may optionally be modified by truncation to form a stronger but less sharp form.

Protrusions 106 may be produced using a wide range of different technologies from a wide variety of different materials. For example, MEMS technology (using wet or dry etching or a combination of the two) may be employed to process a unitary block of silicon (single crystal) or other etchable material to produce the protrusions-plus-substrate structure. Suitable MEMS techniques for forming a wide variety of conical, pyramidal and cylindrical protrusions projecting from a substrate are well known in the art, for example, in the context of microneedle technology. Most preferably, low cost MEMS techniques based primarily on wet etching techniques are used.

Other technologies suitable for forming the skin interface element include injection or micro-injection molding, hot embossing and machining techniques which be used to produce the skin interface element from various polymers or other moldable materials. According to a further option, foils (such as steel, titanium, or other metals or metal alloys) may be processed by cutting (wire cutting, laser cutting, punching or other cutting processes), with or without post cutting processing, to form protrusions 106. Ceramics may also be used. In most preferred implementations, protrusions 106 are integrally formed with substrate 104.

Referring again to FIG. 1, skin interface element 102 is preferably supported relative to a housing 110 via a resilient support 112 which allows vibratory motion of skin interface element 102 without excessive damping from the mass of housing 110 and the user's hand holding the device. In the case of FIG. 1, resilient support 112 is shown as a flexible membrane which performs an additional function of sealing between skin interface element 102 and housing 110 to prevent ingress of dirt and other foreign matter.

Generation of vibration can be achieved using any of a wide range of mechanisms. By way of one preferred but non-limiting example, vibration generating mechanism 108 as illustrated here includes an electric motor 114 driving an eccentric weight 116 about an axis 118. The motor is driven by a power supply 120, typically implemented as one or more battery mounted within housing 110, and is controlled by on/off switch 122. Vibration generating mechanism 108 can thus be implemented cheaply using compact off-the-shelf components such as those employed for vibrating notification in cellular telephones.

The deployment of the vibration generating mechanism and its attachment to the other parts of the device are chosen relative to the micro-protrusions in order to provide a desired form of vibrational motion relative to the skin surface (e.g., orbital motion on the skin, motion perpendicular to the skin, a back and fro motion on the skin, or any combination of these motions). Thus, for example, in the case illustrated here, axis 118 is substantially parallel to the surface of substrate 104, resulting in vibratory motion having a first component parallel to the skin surface and a second (non-zero) component perpendicular to the skin surface. Alternatively, axis 118 may be deployed perpendicular to the surface of substrate 104, resulting in a rotating force vector in a plane of the substrate and a corresponding orbital motion of skin interface element 102. A preferred non-limiting range of frequencies for the vibration generating mechanism is between 50 Hz and 200 Hz, and most preferably, in the range of 140 Hz±25 Hz.

A further feature of certain preferred embodiments of the present invention is illustrated schematically in FIG. 2. FIG. 2 shows a device, generally designated 100′, which is equivalent to device 100 of FIG. 1 except that it features an additional pressure-limiting switch arrangement 124. Pressure-limiting switch arrangement 124 is responsive to contact pressure of skin interface element 102 above a given limit to interrupt operation of the vibration generating mechanism 108. This ensures that contact pressure exerted by the hand of the user does not reach sufficient levels to cause excessive penetration depth, or to lodge protrusions 106 firmly into the tissue, an effect which might lead to excessive damping of vibrations and consequent disruption to the efficacy of the abrasion treatment. This feature is particularly important for medical application (e.g., treatment to increase porosity of the skin to enhance absorption of medication) where relatively longer protrusions may be used and regulation of penetration depth therefore becomes more important.

Structurally, pressure-limiting switch arrangement 124 is shown here implemented as a circuit breaker included in the power supply circuit for vibration generating mechanism 108. The resilient mounting of skin interface element 102 allows for retraction of the skin interface element as a function of contact pressure. By leaving an appropriately chosen gap between the rear end of a shaft 126 of skin interface element 102 and the circuit breaker, a desired threshold of contact pressure can be defined for the cut-out function. Optionally, pressure-limiting switch arrangement 124 may be configured to operate an alarm or buzzer (not shown) if the contact pressure exceeds the defined limit.

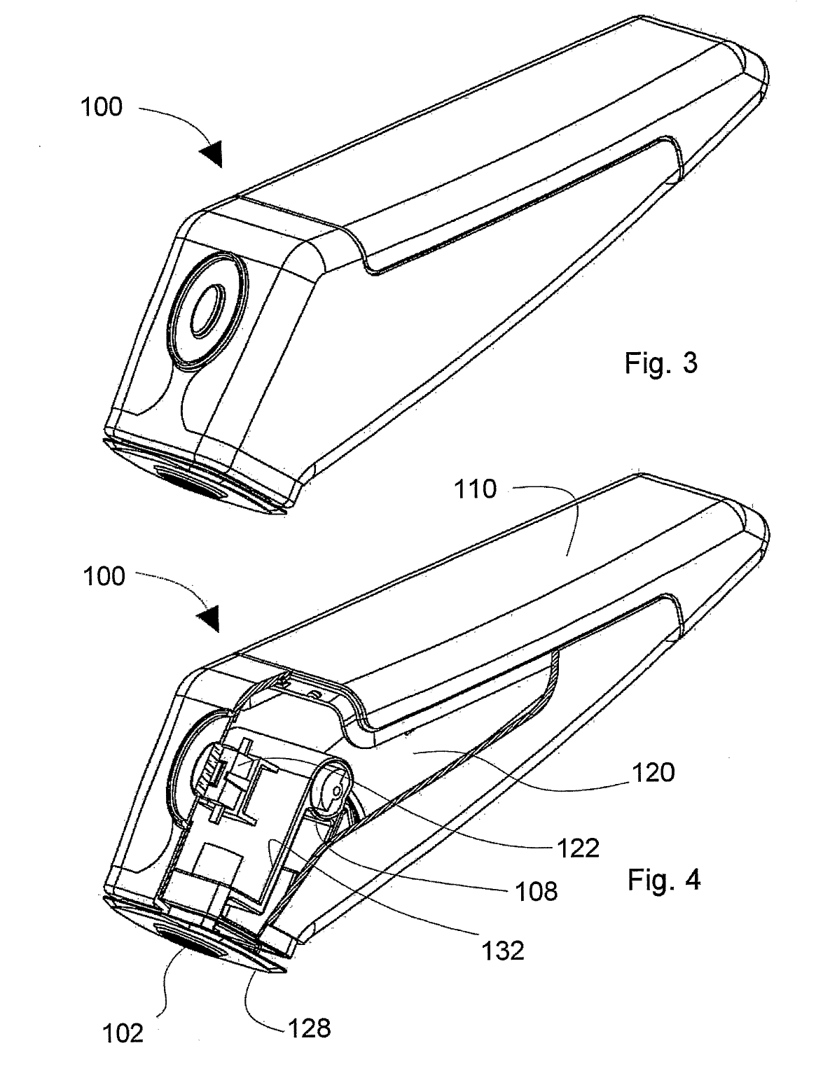

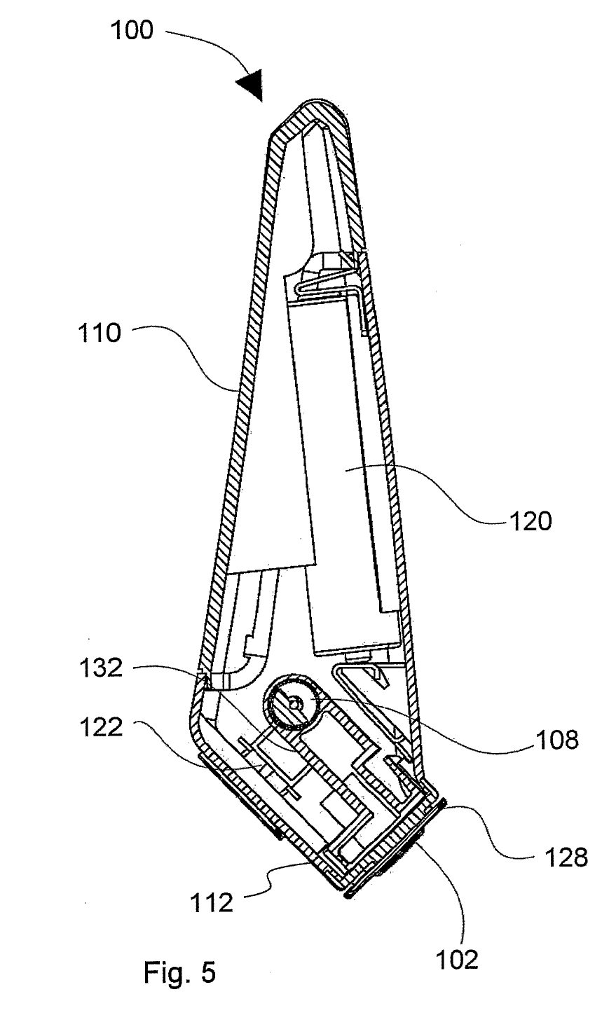

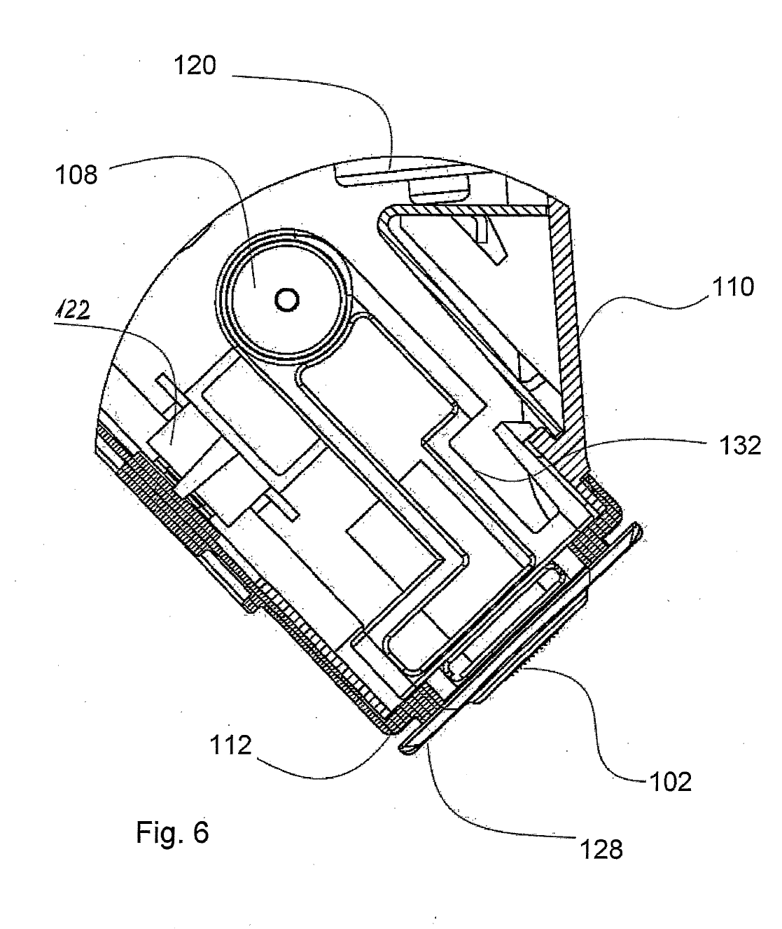

Turning now to FIGS. 3-7, these illustrate one non-limiting practical implementation of device 100. The device shown is essentially identical to the device illustrated schematically in FIG. 1, with equivalent elements labeled similarly. However, a few additional features appear here which were omitted for clarity from the schematic representation of FIG. 1. These features will now be addressed.

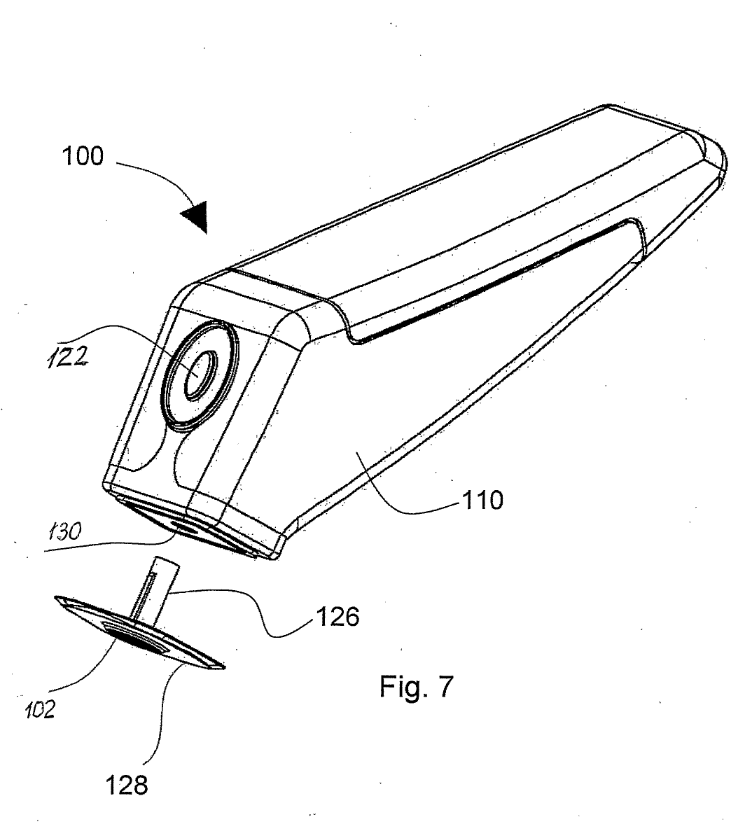

One particularly preferred feature, best illustrated in FIG. 7, is that skin interface element 102 is implemented as a replaceable, disposable insert. This facilitates proper hygiene, enabling all parts of the device coming in contact with the treated area of skin to be new and clean for each use while avoiding unnecessary costs of replacing other parts of the device. Most preferably, substrate 104 is provided with an additional shield 128 which covers substantially the entire front surface of the housing 110, thereby preventing contact of any non-disposable part of the device with the treated skin during use. To accommodate this design, resilient support 112 is here implemented as a peripheral bridging portion which supports a central assembly 132 including vibration generating mechanism 108 and a socket 130 for receiving shaft 126. In the particularly preferred implementation shown here, resilient support 128 further extends to the region of on/off switch 122 in order to provide a sealed cover for the switch.

Housing 110 and other parts of device 100 are preferably formed from common thermoplastic polymers suitable for injection molding, such as for example ABS (Acrylonitrile Butadiene Styrene). Resilient support 108 may be formed of any suitable resilient material, including but not limited to, natural or artificial rubber or silicone.

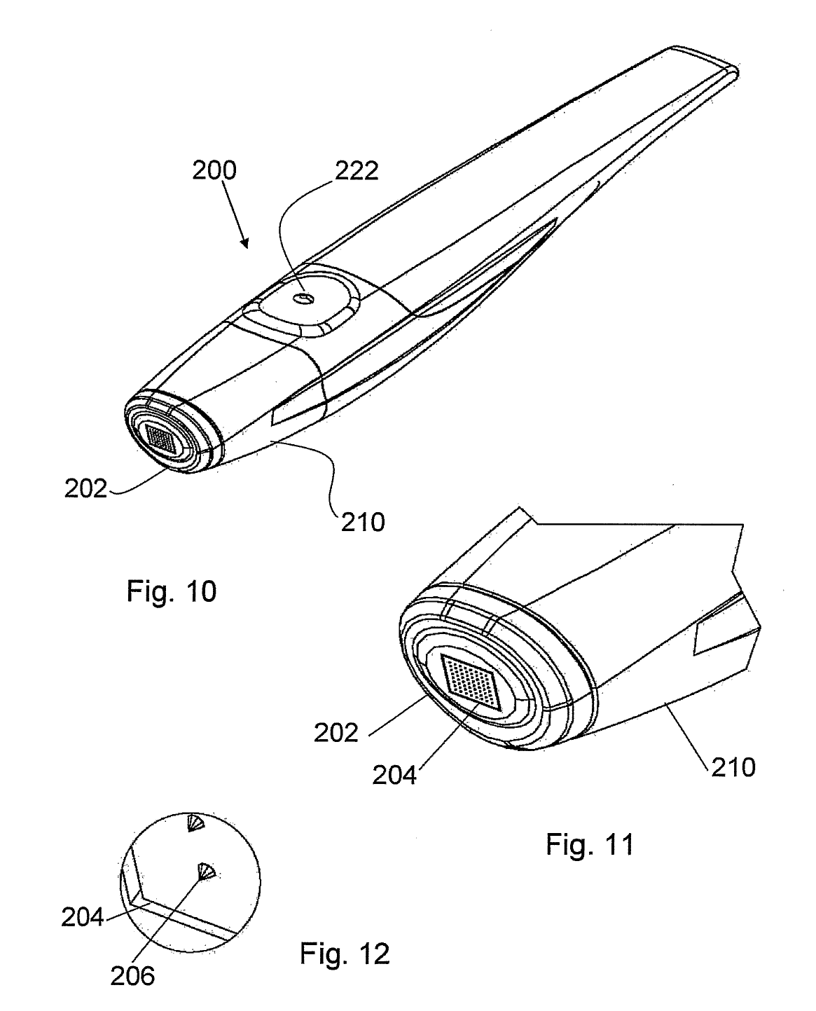

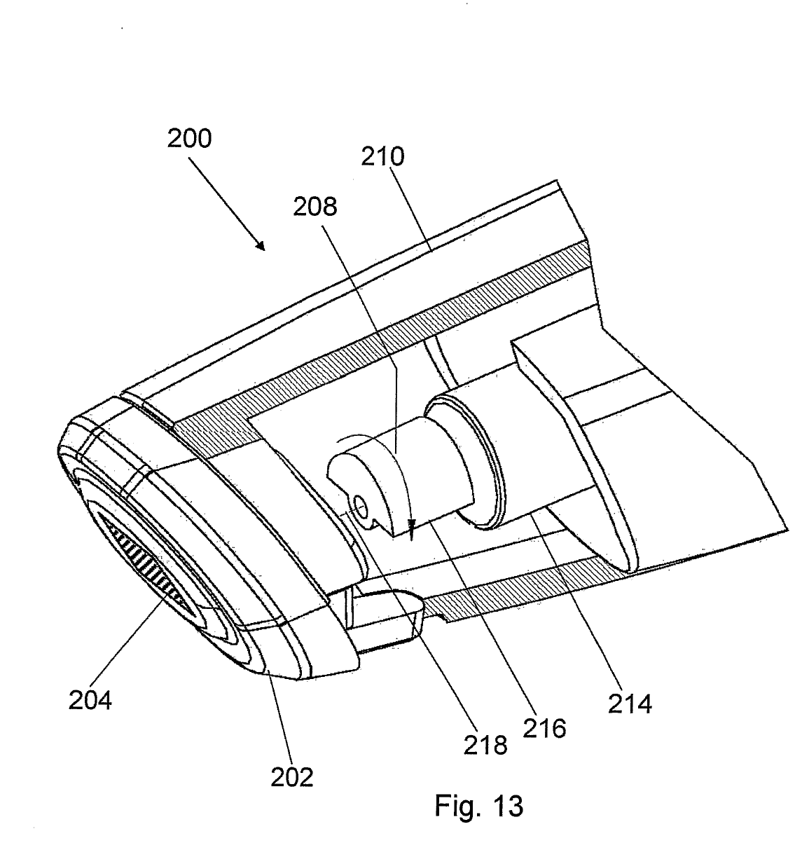

Turning now to the remaining FIGS. 10-13, these illustrate an alternative implementation of the device of the present invention, generally designated 200. Device 200 is structurally and functionally analogous to device 100, and equivalent elements are labeled similarly with addition of 100 to their reference numerals. Device 200 differs from device 100 primarily in that motor 214 is here deployed to rotate eccentric weight 216 about an axis 218 substantially perpendicular to the plane of substrate 204, as best seen in FIG. 13. As a result, the vibratory motion generated by the device is primarily orbital motion in the plane of skin contact.

Additionally, the slim and flat-tailed form of housing 210 has been found particularly ergonomically advantageous for facial applications of the device. In all other respects, the structure and operation of device 200 will be fully understood by analogy to the structure and operation of device 100 as described above.

Finally, it should be noted that the present invention may be used to advantage in a wide range of cosmetic and medical application. By way of non-limiting examples, various application procedures could be employed in combining the device with a specific active (cream, gel, solution or the like). For example: cosmetic or dermatologic pre-treatment (skin treatment prior to applying the active composition), cosmetic or dermatologic post treatment (using the device after the cream, paste or solution were applied on treated site), and cosmetic or dermatologic treatment: cream and projections applied concurrently. It should be noted that particularly preferred implementations of the present invention relate to general purpose devices which may be used with various different treatment compositions, and wherein the device itself typically does not store or apply the composition.

Any cosmetic and pharmaceutical agents may be incorporated or delivered with the abovementioned systems to enhance the therapeutic effects of those cosmetic and pharmaceutical agents to improve cosmetic conditions or to alleviate the symptoms of dermatologic disorder. Cosmetic and pharmaceutical agents include those that improve or eradicate age spots, keratoses and wrinkles; analgesics; anesthetics; antiacne agents; antibacterials; antiyeast agents; antifungal agents; antiviral agents; antidandruff agents; antidermatitis agents; antipruritic agents; antiemetics; antimotion sickness agents; antiinflammatory agents; antihyperkeratolytic agents; antidryskin agents; antiperspirants; antipsoriatic agents; antiseborrheic agents; hair conditioners and hair treatment agents; antiaging and antiwrinkle agents; antiasthmatic agents and bronchodilators; sunscreen agents; antihistamine agents; skin lightening agents; depigmenting agents; vitamins; corticosteroids; tanning agents; hormones; retinoids; topical cardiovascular agents and other dermatologicals.

Some examples of cosmetic and pharmaceutical agents are clotrimazole, ketoconazole, miconazole, griseofulvin, hydroxyzine, diphenhydramine, pramoxine, lidocaine, procaine, mepivacaine, monobenzone, erythromycin, tetracycline, clindamycin, meclocycline, hydroquinone, minocycline, naproxen, ibuprofen, theophylline, cromolyn, albuterol, retinoic acid, 13-cis retinoic acid, hydrocortisone, hydrocortisone 21-acetate, hydrocortisone 17-valerate, hydrocortisone 17-butyrate, betamethasone valerate, betamethasone dipropionate, triamcinolone acetonide, fluocinonide, clobetasol propionate, benzoyl peroxide, crotamiton, propranolol, promethazine, vitamin A palmirate and vitamin E acetate.

It will be appreciated that the above descriptions are intended only to serve as examples, and that many other embodiments are possible within the scope of the present invention as defined in the appended claims.

What is claimed is:

1. A device for superficial abrasive treatment of the skin comprising:
(a) a skin interface element including a substrate provided with a plurality of protrusions, said protrusions projecting to a height above said substrate of no greater than 200 microns; and
(b) a vibration generating mechanism mechanically linked to said skin interface element so as to generate vibratory motion of said skin interface element.

2. The device of claim 1, wherein said protrusions have a height above said substrate of between about 20 microns and about 100 microns.

3. The device of claim 1, wherein said protrusions are arranged in a two-dimensional array.

4. The device of claim 1, wherein said protrusions have a shape selected from the group comprising: pyramids, cones and rods.

5. The device of claim 1, wherein said protrusions are integrally formed with said substrate.

6. The device of claim 1, wherein said protrusions and said substrate are formed from a single crystal of material.

7. The device of claim 1, wherein said protrusions and said substrate are formed from a unitary block of material processed primarily by wet etching techniques.

8. The device of claim 1, wherein said protrusions are formed from a material selected from the group consisting of: silicon, a polymer, a metal, a metal alloy, and a ceramic material.

9. The device of claim 1, wherein said vibration generating mechanism includes a motor configured for rotating an eccentric weight about an axis.

10. The device of claim 1, wherein said vibration generating mechanism is configured to generate vibratory motion corresponding to an orbital motion in a plane of said substrate.

11. The device of claim 1, wherein said vibration generating mechanism is configured to generate vibratory motion having a non-zero component perpendicular to a plane of said substrate.

12. The device of claim 1, wherein said vibration generating mechanism is configured to generate vibratory motion having a frequency in the range between 50 Hz and 200 Hz.

13. The device of claim 1, wherein said vibration generating mechanism is configured to generate vibratory motion having a frequency in the range of 140 Hz±25 Hz.

14. The device of claim 1, further comprising a pressure-limiting switch arrangement, associated with said skin interface element and responsive to contact pressure of said skin interface element above a given limit to interrupt operation of said vibration generating mechanism.

15. The device of claim 1, further comprising a housing, wherein said skin interface element is resiliently mounted relative to said housing, and wherein said vibration generating mechanism is mechanically linked to said skin interface element so as to generate vibratory motion of said skin interface element relative to said housing.

16. A method for superficial abrasive treatment of the skin comprising the steps of:
(a) bringing into contact with the skin a skin interface element including a substrate provided with a plurality of protrusions, said protrusions projecting to a height above said substrate of no greater than 200 microns; and
(b) employing a vibration generating mechanism mechanically linked to said skin interface element to generate vibratory motion of said skin interface element so that said protrusions perform superficial abrasive treatment of the skin.

17. The method of claim 16, wherein said protrusions have a height above said substrate of between about 20 microns and about 100 microns.

18. The method of claim 16, wherein said protrusions are arranged in a two-dimensional array.

19. The method of claim 16, wherein said protrusions have a shape selected from the group comprising: pyramids, cones and rods.

20. The method of claim 16, wherein said vibratory motion is generated by a motor configured for rotating an eccentric weight about an axis.

21. The method of claim 16, wherein said vibratory motion corresponds to an orbital motion in a plane of said substrate.

22. The method of claim 16, wherein said vibratory motion has a non-zero component perpendicular to a plane of said substrate.

23. The method of claim 16, wherein said vibratory motion has a frequency in the range between 50 Hz and 200 Hz.

24. The method of claim 16, wherein said vibratory motion has a frequency in the range of 140 Hz±25 Hz.

25. The method of claim 16, further comprising interrupting operation of said vibration generating mechanism when a contact pressure between said skin interface element and the skin exceeds a given limit.
